(19) United States
(12) Patent Application Publication
(10) Pub. No.: US 2023/0072066 A1
(43) Pub. Date: Mar. 9, 2023

(54) Choroidal Imaging

(71) Applicant: BROADSPOT IMAGING CORP, Emeryville, CA (US)

(72) Inventors: Tushar M. Ranchod, Dunfermline (GB); Clark Pentico, Dunfermline (GB); Andre E. Adams, Dunfermline (GB); Jacobson A. Benjamin, Dunfermline (GB)

(73) Assignee: OPTOS PLC, Dunfermline (GB)

(21) Appl. No.: 17/750,846

(22) PCT Filed: Nov. 24, 2020
(86) PCT No.: PCT/US20/62099

§ 371 Date: May 23, 2022

Related U.S. Application Data
(60) Provisional application No. 62/940,170, filed on Nov. 25, 2019.

(51) Int. Cl. A61B 3/10; G06T 5/00; G06T 5/50; G06T 7/13; G06V 10/141; A61B 3/12

(52) U.S. Cl. CPC A61B 3/102; G06T 5/004; G06T 5/50; G06T 7/13; G06V 10/141; A61B 3/12; G06T 2207/20081; G06T 2207/20221; G06T 2207/30041

(57) ABSTRACT

A method may include illuminating a region of a choroid of an eye of a patient with off-axis illumination from a first imaging channel, the first imaging channel being off-axis with respect to an axis of a focus of the eye. The method may also include capturing an image of the choroid, where the off-axis illumination from the first imaging channel is off-set within the first imaging channel from the image sensor. A second off-axis illumination from a second imaging channel that is off-axis from both the first imaging channel and the off-axis illumination may illuminate the same or a different region of the choroid. The captured image of the choroid may be provided to a machine learning system. Indices associated with the image to may be identified based on an output of the machine learning system.

### FIELD

The application relates generally to devices and processing used in choroidal imaging.

### BACKGROUND

Imaging of the choroidal vessels in the eye is a challenging endeavor. For example, the pigment in the retina and/or retinal pigment epithelium (RPE) may shield or mask the choroidal vessels. Additionally or alternatively, reflection of illumination off of the retina and/or RPE may prevent obtaining a clear image of the choroidal vessels.

The subject matter claimed herein is not limited to embodiments that solve any disadvantages or that operate only in environments such as those described above. Rather, this background is only provided to illustrate one example technology area where some embodiments described herein may be practiced.

### SUMMARY

One or more embodiments of the present disclosure may include a method that includes illuminating a region of a choroid of an eye of a patient with off-axis illumination from a first imaging channel and illumination from a second imaging channel that is off-axis from the first imaging channel. The method may also include capturing an image of the choroid using an image sensor in the first imaging channel, where the off-axis illumination from the first imaging channel is off-set within the first imaging channel from the image sensor. The method may additionally include identifying one or more indices based on the image of the choroid. The method may also include providing the captured image and the one or more indices to a machine learning system.

### BRIEF DESCRIPTION OF FIGURES

Example embodiments will be described and explained with additional specificity and detail through the use of the accompanying drawings in which:

FIG. 1A illustrates an example channel of a broad-field multi-channel imaging device;

FIG. 1B illustrates an example of illumination and imaging rays of the broad-field multi-channel imaging device of FIG. 1A;

FIG. 2 illustrates an example broad-field image overlaid with coverage of a typical fundus camera and optical coherence tomography (OCT);

FIGS. 3A and 3B illustrate two example views of an eye captured with two different imaging devices;

FIG. 3C illustrates a close-up view of the image of FIG. 3A;

FIGS. 4A and 4B illustrate two additional example views of an eye captured with two different imaging devices;

FIGS. 5A and 5B illustrate two additional example views of an eye captured with two different imaging devices;

FIG. 6 illustrates an additional example view of an eye captured with a broad-field multi-channel imaging devices;

FIG. 7 is a flowchart of an example method 700 of imaging a choroid of an eye of a patient; and

FIG. 8 illustrates an example computing system.

### DESCRIPTION OF EMBODIMENTS

The present disclosure relates to, inter alia, the use of a broad-field multi-channel imaging device to capture images of internal regions of the eye. Certain imaging techniques and imaging processing techniques may be used to provide a robust and expansive view of the choroid and/or the choroidal vasculature. For example, certain wavelengths of illumination, certain depths of focus, and/or certain image processing techniques may highlight the choroidal vasculature in an improved manner compared to previous approaches to capturing images of the choroidal vasculature. As another example, the use of a multi-channel imaging device where the illumination and imaging rays are off-axis may permit greater imaging of the choroid. Because the illumination is off-axis and/or polarized, using such an imaging device may reduce direct reflections from the retinal surface and/or RPE, allowing greater visibility of the choroidal vessels, particularly in the macular region but also into the far periphery, when compared to existing imaging devices.

The choroid may include the tissue between the retina and the sclera in the eye. The choroid may include multiple layers of varying sizes of vasculature, and connecting tissue/membranes, including the Haller's layer with larger diameter blood vessels, Sattler's layer with medium diameter blood vessels, Choriocapillaris with capillaries, and Bruch's membrane as the innermost layer of the choroid. The choroid is typically between around 0.1 millimeters (mm) and 0.2 mm thick, with the thickest region at the far rear of the eye. Because of the location of the choroid below the retina and the RPE, the choroid and the choroidal vasculature is difficult to image. In particular, the retina and the RPE absorb most of the light in the 200-600 nanometer (nm) range. Additionally, the fluid within the eye absorbs much of the light in the 900-1000 nm range. For individuals with light pigmented eyes (such as blue eyes), a traditional fundus camera may capture a certain amount of choroidal vasculature due to a decreased amount of absorbance and/or reflectance by the retina and RPE. However, even if targeted wavelengths of illumination are used in typical fundus cameras, much of the choroidal vasculature may be obscured by the retina and RPE.

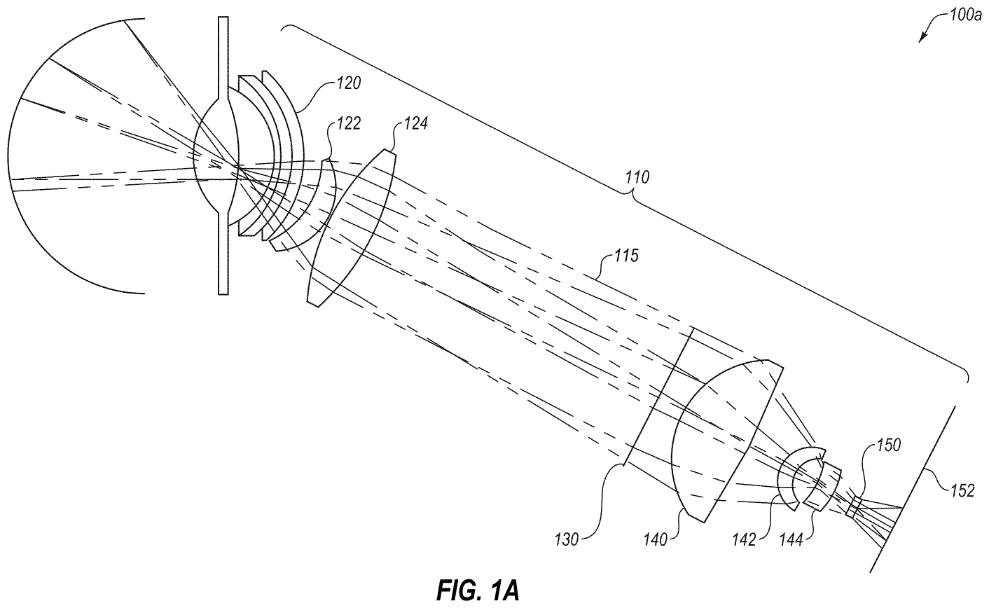

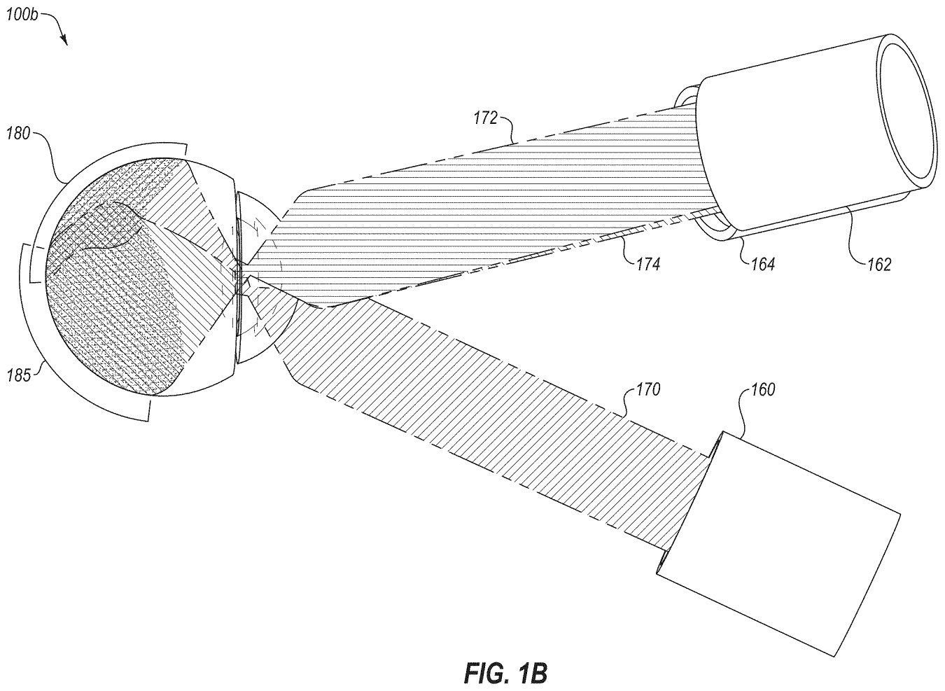

FIG. 1A illustrates one example of a broad-field multi-channel imaging device 100a, and FIG. 1B illustrates an example of illumination and/or imaging rays associated with such a device 100b. Such a device may be described in U.S. Non-Provisional application Ser. No. 16/698,024, the entire disclosure of which is incorporated by reference herein in its entirety. However, such a device is only one example of such a device and the embodiments described herein are not limited to images captured using the device illustrated in FIGS. 1A and/or 1B. Rather, the present disclosure may be applicable to any device using multiple imaging channels.

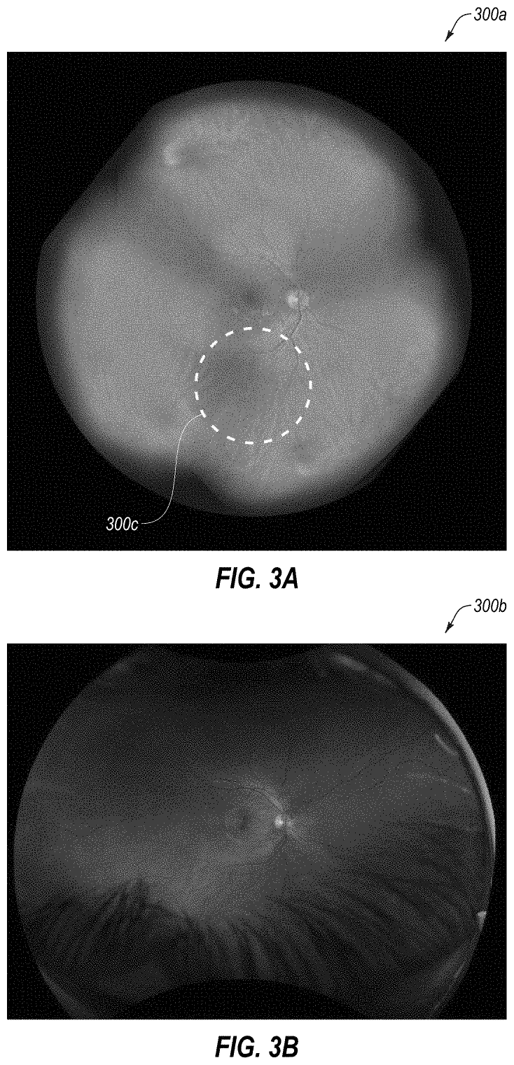

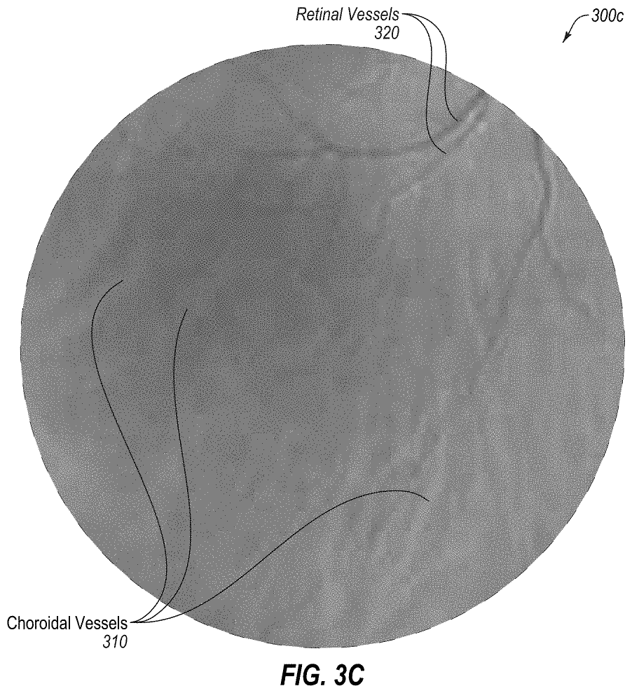

FIG. 1A illustrates an example channel 110 of a broad-field multi-channel imaging device 100a, in accordance with one or more embodiments of the present disclosure. The imaging device may include multiple imaging channels 110. The channel 110 may include optical traces 115, a glass window 120, one or more glass lenses 122 and 124, one or more polarizers 130, one or more relay lenses 140, 142, and 144, a camera aperture 150, and one or more camera sensors 152. The channel 110 may be off-axis from the central axis of the device (e.g., an axis coming directly out from the pupil of the eye). Stated another way, the channel 110 may work in conjunction with other similar channels that are oriented at an angle relative to the eye and the multiple channels work together to form an image of the eye. For example, the channels 110 may image overlapping regions of the eye such that a large portion of the internal of the eye may be imaged. Various examples of images captured from such an imaging device are illustrated in FIGS. 3A, 4A, 5A and 6.

FIG. 1B illustrates an example of illumination and imaging rays of the broad-field multi-channel imaging device of FIG. 1A, in accordance with one or more embodiments of the present disclosure. FIG. 1B illustrates a multi-channel imaging system 100b including a first imaging channel 160, a second imaging channel 162, a third imaging channel 164, first optical traces 170, second optical traces 172, and third optical traces 174 for imaging a first region 180 and a second region 185. An example of such a device, including the operation of the imaging channels and illumination sources, is described in greater detail in U.S. Non-Provisional application Ser. No. 16/698,024. The imaging device 100b may be any imaging device with illumination that is off-axis from the image-capturing pathway with multiple imaging channels that are off-axis. For example, many imaging devices use an imaging pathway that is co-axial with the pupil of the eye and use illumination along a similar axis, such as illumination about the periphery of the imaging pathway. The imaging device 100b may include illumination that is angled and off-set relative to the imaging pathway. In some embodiments, that illumination may come from a different imaging channel all together, from the same imaging channel but offset and angled relative to the imaging pathway, and/or combinations thereof. For example, one region relatively aligned with the imaging pathway may be illuminated by another imaging channel, and peripheral regions may be illuminated by illumination sources offset but in the same imaging channel as the imaging device. Additionally, the imaging channels may be off-axis from an axis of the focus of the eye. In these and other embodiments, the imaging channels may be off-axis from the axis of the focus of the eye, and the illumination for the imaging channel may be off-axis from the imaging channel.

In some embodiments, by utilizing off-axis illumination there may be less illumination reflected directly by the retina and/or the RPE such that the imaging device with the off-axis illumination may better capture the choroidal vasculature.

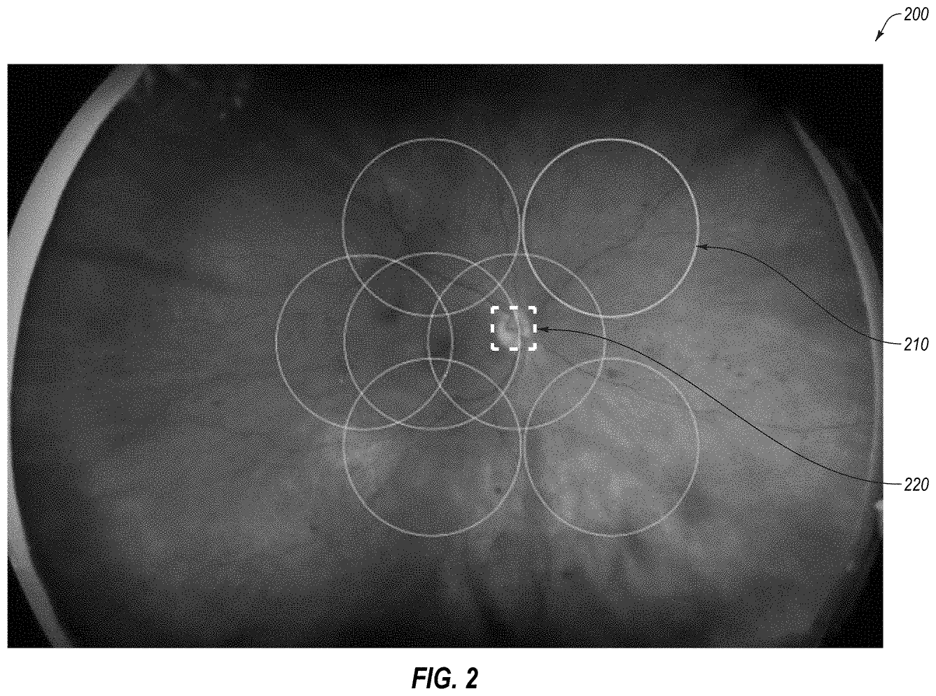

FIG. 2 illustrates an example broad-field image 200 overlaid with coverage of a typical fundus camera and optical coherence tomography (OCT), in accordance with one or more embodiments of the present disclosure. The overall image 200 may represent the scope of coverage with a broad-field multi-channel imaging device in accordance with the present disclosure and as described in U.S. Non-Provisional application Ser. No. 16/698,024. For example, the solid circle 210 may represent a region captured by a typical fundus camera (e.g., approximately fifty degrees). As another example, the dashed square 220 may represent a region captured by OCT. As can be seen by observing the solid circles compared to the broad-field image 200, the broad-field image 200 captures a much larger region of the eye including a wider field of view when compared to that captured by a typical fundus camera or OCT. By capturing the larger region, patterns and features of the entire or nearly the entire choroid may be imaged and/or analyzed, while an image of the size of the solid circle 210 or the dashed square 220 may provide information for only a very small and specific region of the choroid. While the specific view obtainable by a typical fundus camera and/or OCT may be useful for some purposes, the wider macro-level view may be useful in other circumstances and for other purposes. For example, the lateral slice view of OCT may be used to measure the thickness of the choroid, but a lateral slice view in one location cannot provide the macro-level view illustrated in FIGS. 3A, 4A, 5A, and/or 6. In some embodiments, a macro-level image may capture at least sixty percent of the choroid, at least 70% of the choroid, at least 75% of the choroid, at least 80% of the choroid, at least 85% of the choroid, at least 90% of the choroid, at least 95% of the choroid, and/or 100% of the choroid. In addition, AI algorithms may obtain at least some key information from more diffuse or non-central aspects of fundus photographs. For example, ultra-wide-field cameras such as those consistent with the present disclosure may provide useful information for AI purposes.

FIGS. 3A and 3B illustrate two example views of an eye, illustrating different views captured with two different imaging devices. The image 300a illustrated in FIG. 3A is captured via an imaging device 100 as illustrated in FIG. 1B. The image 300b illustrated in FIG. 3B is the same eye captured via a wide-field scanning laser opthalmoscopy (SLO) imaging device. FIG. 3C illustrates a close-up view 300c of a portion of the image of FIG. 3A. The close-up view 300c of FIG. 3C illustrates both choroidal vessels 310 and retinal vessels 320. As can be seen, a wide view of the choroidal vessels 310 may be seen in the image 300a. Thus, as illustrated in FIG. 3A, a macro-level view of the choroid from a frontal view is obtained using the imaging device 100 as illustrated in FIG. 1. The macro-level view of information allows a broader view into the vasculature of the choroid as compared to a typical fundus camera (e.g., the solid circle 210 of FIG. 2) or OCT (e.g., the dashed square 220 of FIG. 2).

In some embodiments, different views of the choroid may be captured by processing of the macro-level view. For example, an image such as the image 300a may have certain wavelengths emphasized or filtered out when rendering the image. In doing so, certain portions of the choroid may be more readily visible. For example, longer wavelengths (e.g., red, near infrared, infrared, etc.) may be emphasized when rendering an image to emphasize the choroidal vasculature. In these and other embodiments, a wide-spectrum light source may be used for illumination, such as a bright white light emitting diode (LED) illumination source that also covers at least a portion of the infrared spectrum. After capturing the data from an imaging sensor, when rendering the data on a display, certain wavelengths may be emphasized or filtered. For example, when rendering the RBG values at a given pixel, the red values may be emphasized and the blue and green values may be diminished.

In some embodiments, image processing may be performed on the data captured by the image sensor. One such image processing technique includes a sharpening technique. For example, an unsharp mask may be applied to the image to enhance the brightness difference along detected edges in the image. In such image processing, a sharpening radius to detect edges may be used that corresponds with choroidal vessels generally, or to the layer of choroidal vessels to be viewed/analyzed. For example, different sharpening radiuses of edges may be used to highlight a target layer of the choroid, the target layer of the choroid having a radius of a target size, such as the Haller's layer (a larger radius for larger vessels) and/or the Choriocapillaris (a smaller radius for capillaries).

In some embodiments, a series of images may be captured. In some embodiments, the series of images may be at a common focus depth and/or wavelength of illumination. In these and other embodiments, the series of images may be combined into a video. Such a video may permit capture of flow in the choroidal vessels, such as by visualizing the pulse or heartbeat of the user. In some embodiments, the series of images may be captured with varying depths of focus. For example, a series of images may be captured with the focus at multiple depths of the choroid, such that different layers of vasculature of the choroid are in focus and can be seen more clearly in the different images (e.g., a first image may be at a depth to view the Choriocapillaris in most focus, a second image may be at a depth to view the Haller's layer in most focus).

In some embodiments, various indices of the choroid may be captured or rendered via computer processing of the images of the choroid. One example of such indices may include an average choroidal vascular caliber, either across all choroidal vessels or for subsets of choroidal vessels defined by ranges of choroidal vessel lumen diameter or location in the image. For example, the outer diameter (e.g., as the vessel caliber) of all vessels in a region of the image may be determined and a count of the number of vessels may be used to determine the average caliber of the vessels. Another example of such indices may include an average choroidal vascular tortuosity, either across all choroidal vessels or for subsets of choroidal vessels defined by ranges of choroidal vessel lumen diameter or location in the image. For example, the length of a given vessel in a set amount of distance may be measured and set as a ratio (e.g., Lᵥₑₛₛₑₗ/L_{distance} may yield a numerical value of tortuosity) to determine the tortuosity of the given vessel, and the average tortuosity for all choroidal vessels in a region or the entire image. An additional example of such indices may include a ratio of choroidal vascular caliber to retinal vascular caliber, either across all retinal and choroidal vessels or some subset. Another example of such indices may include a ratio of choroidal vascular tortuosity to retinal vascular tortuosity, either across all retinal and choroidal vessels or some subset. Another example of such indices may include a categorization based on choroidal branching patterns (e.g., number of branches in unit of distance, directionality of branching, designation among a set number of predefined choroidal vascular patterns, etc.) or choroidal vascular density (e.g., number of vessels in unit of distance, etc.). In these and other embodiments, these various indices may be determined automatically or by manual identification. Any other indices may be used and are contemplated in conjunction with the present disclosure. In some embodiments, a particular region of the macro-level view of the choroid may be manually selected for analysis. For example, a region may be selected for determining choroidal vascular caliber and/or retinal vascular caliber.

In some embodiments, one or more of the indices of the present disclosure may be used on an absolute level (e.g., may be determined as a single event or analysis to facilitate determination of a patient's systemic or ocular condition, with or without clinical data or demographic data). For example, a patient may come in for a routine eye exam and image(s) of the choroid may be captured as part of the eye exam, and one or more of these indices may be determined from the image(s) of the choroid. Additionally or alternatively, such indices may be used to calculate changes before and after interventions to understand the effect of the intervention. For example, images may be taken before and after dialysis with removal of substantial amounts of fluid from the patient's body during dialysis, and images before and after dialysis may help determine the efficacy of the fluid removal in reducing fluid overload based on the indices. As another example, isolated images before the intervention may help determine the degree of fluid status and therefore how much fluid is to be removed during dialysis. In some embodiments, other conditions or interventions may be analyzed or considered in light of the choroidal indices.

In some embodiments, the indices and/or the images of the choroidal vasculature themselves may be provided to a machine learning system. In these and other embodiments, the machine learning system may utilize the indices and/or images to identify patterns or features associated with the patient that human vision alone may not recognize. For example, machine learning applied to images captured using traditional fundus cameras have been shown to identify gender, smoking status, etc. with a high degree of accuracy even though human analysis is unable to identify such characteristics. In these and other embodiments, the machine learning system may identify correlations, patterns, etc. discernable via the choroidal images. For example, a set of training data may be provided to a machine learning system that includes one or more choroidal images and/or indices for an individual along with other factors related to the individual (e.g., disease conditions, health habits, genetic characteristics, etc.) and the machine learning system may analyze and determine patterns and correlations between the factors of individuals and the aspects of the choroidal images and/or indices. As another example, various AI algorithms may be trained based on sets of such images (preferably large sets) to specifically identify specific diseases or endpoint measures using choroidal parameters based on identified correlations. Such correlations may relate to various disease conditions, health habits, fluid levels, genetic disorders or predispositions, etc. For example, risk assessments for specific diseases based on a cluster of presenting symptoms combined with choroidal imaging, for example the risk of cerebrovascular accident (e.g., stroke) for somebody presenting with stroke-like (or transient ischemic attack-like) symptoms may facilitate a determination of how urgently an intensive workup is required for the individual with the symptoms.

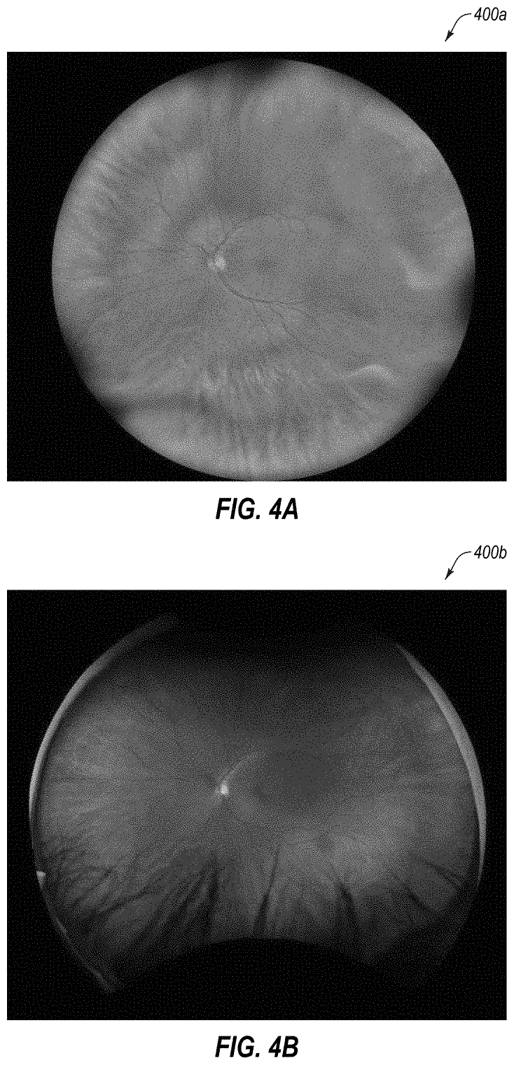

FIGS. 4A and 4B illustrate two additional example images 400a and 400b of an eye captured with two different imaging devices. The imaging devices used to capture the images 400a in FIG. 4A and 400b in FIG. 4B may be the same or similar to those used to capture the images 300a in FIG. 3A and 300b in FIG. 3B, respectively.

The image 400a illustrates another image of another eye as compared to the image 300a. Additionally, FIG. 4A illustrates an image in which the red tones of an image have been enhanced to a greater degree than those illustrated in FIG. 3A.

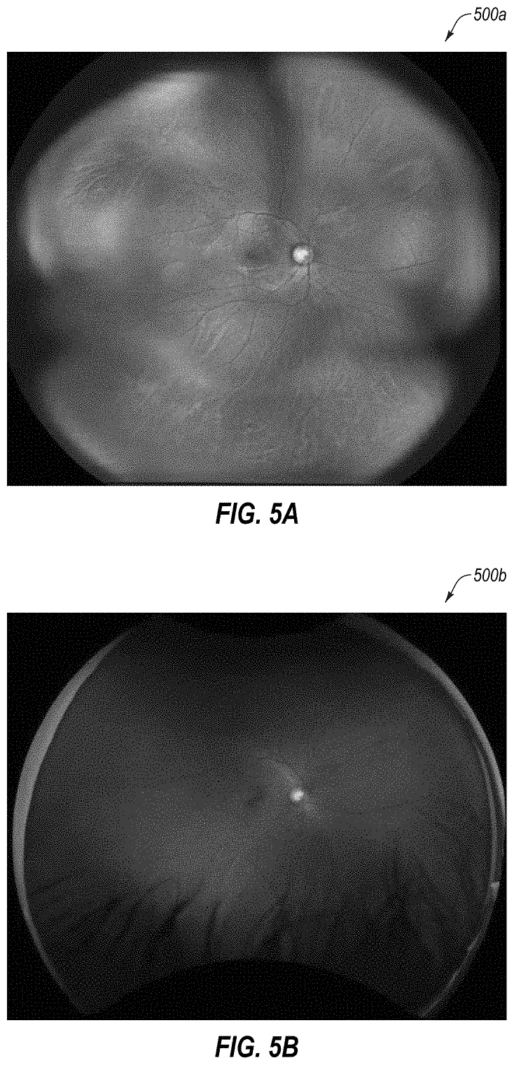

FIGS. 5A and 5B illustrate two additional example images 500a and 500b of an eye captured with two different imaging devices, in accordance with one or more embodiments of the present disclosure. The imaging devices used to capture the images 500a in FIG. 5A and 500b in FIG. 5B may be the same or similar to those used to capture the images 300a in FIG. 3A and 300b in FIG. 3B.

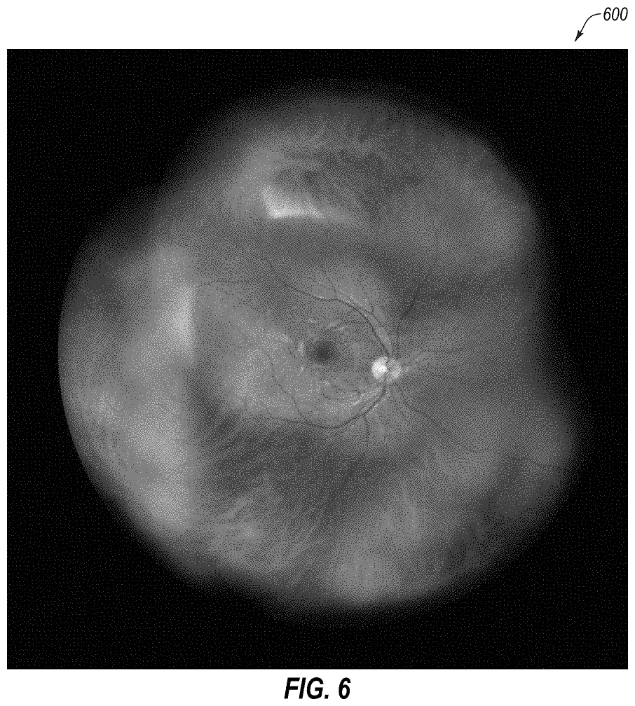

FIG. 6 illustrates an additional example image 600 of an eye captured with a broad-field multi-channel imaging devices. The imaging device used to capture the image 600 in FIG. 6 may be the same or similar to that used to capture the image 300a in FIG. 3A.

The imaging device used to capture the images 300a, 400a, 500a, and/or 600 (e.g., the imaging device 100 of FIG. 1B) may be significantly less expensive than the imaging device used to capture the images 300b, 400b, and/or 500b. For example, the imaging devices used to capture the images 300b, 400b, and/or 500b may require expensive lasers of multiple wavelengths with expensive and extensive lens systems. Additionally, such a device may require a large area and be mounted on a desk. In contrast, the imaging device used to capture the images 300a, 400a, 500a, and/or 600 (e.g., the imaging device 100 of FIG. 1B) may be much smaller and utilize much less expensive illumination (e.g., white LED vs. multi-wavelength laser systems). Additionally, the imaging device used to capture the images 300a, 400a, 500a, and/or 600 (e.g., the imaging device 100 of FIG. 1B) may enjoy a much smaller form factor, including a hand-held device or an easily-movable and/or mountable device.

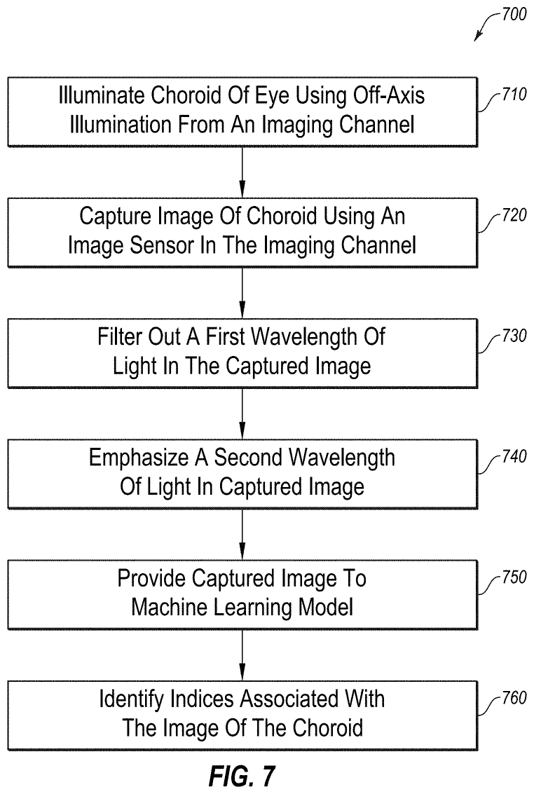

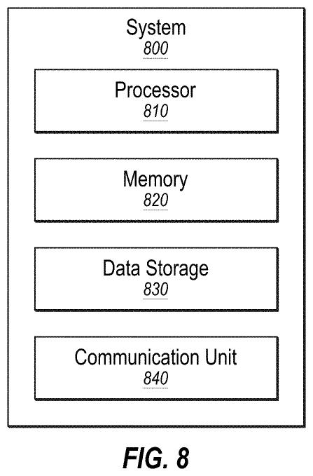

FIG. 7 is a flowchart of an example method 700 of imaging a choroid of an eye of a patient according to at least one embodiment described in the present disclosure. The method 700 may be performed by any suitable system, apparatus, or device. For example, the imaging device illustrated in FIGS. 1A/1B and/or a computing device such as that illustrated in FIG. 8 may perform one or more of the operations associated with the method 700. Although illustrated with discrete blocks, the steps and operations associated with one or more of the blocks of the method 700 may be divided into additional blocks, combined into fewer blocks, or eliminated, depending on the particular implementation.

The method 700 may begin at block 710, where the choroid of the eye of the patient may be illuminated using off-axis illumination from an imaging channel. The choroid of the eye may be illuminated as described above in relation to FIGS. 1A and 1B.

At block 720, an image of the choroid may be captured using an image sensor in the imaging channel. The image of the choroid may be captured as described above in relation to FIGS. 1A and 1B.

At block 730, a first wavelength of light may be filtered out of the captured image. The first wavelength of light may be filtered out of the captured image as described above in relation to FIGS. 3A and 3B.

At block 740, a second wavelength of light may be emphasized in the captured image. The second wavelength of light may be emphasized in the captured image as described above in relation to FIGS. 3A and 3B.

At block 750, the captured image may be provided to a machine learning model. The captured image may be provided to a machine learning model as described in the present disclosure.

At block 760, one or more indices associated with the image of the choroid may be identified. The one or more indices associated with the image of the choroid may be identified as described in the present disclosure.

Modifications, additions, or omissions may be made to the method 700 without departing from the scope of the disclosure. For example, the designations of different elements in the manner described is meant to help explain concepts described herein and is not limiting. Further, the method 700 may include any number of other elements or may be implemented within other systems or contexts than those described.

FIG. 8 illustrates an example computing system 800, according to at least one embodiment described in the present disclosure. The computing system 800 may include a processor 810, a memory 820, a data storage 830, and/or a communication unit 840, which all may be communicatively coupled. Any or all of the operations of the method 700 of FIG. 7 may be implemented by a computing system consistent with the computing system 800. As another example, a computing system such as the computing system 800 may be coupled to and/or part of the imaging device illustrated in FIGS. 1A and 1B.

Generally, the processor 810 may include any suitable special-purpose or general-purpose computer, computing entity, or processing device including various computer hardware or software modules and may be configured to execute instructions stored on any applicable computer-readable storage media. For example, the processor 810 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 8, it is understood that the processor 810 may include any number of processors distributed across any number of network or physical locations that are configured to perform individually or collectively any number of operations described in the present disclosure. In some embodiments, the processor 810 may interpret and/or execute program instructions and/or process data stored in the memory 820, the data storage 830, or the memory 820 and the data storage 830. In some embodiments, the processor 810 may fetch program instructions from the data storage 830 and load the program instructions into the memory 820.

After the program instructions are loaded into the memory 820, the processor 810 may execute the program instructions, such as instructions to perform any steps associated with method 700 of FIG. 7. For example, the processor 810 may obtain instructions regarding obtaining training code, extracting features from the training code, and/or mapping the extracted features to natural language code vectors.

The memory 820 and the data storage 830 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 810. For example, the memory 820 and/or the data storage 830 may store identified indices (such as the one or more indices identified at block 760 of FIG. 7). In some embodiments, the computing system 800 may or may not include either of the memory 820 and the data storage 830.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 810 to perform a certain operation or group of operations.

The communication unit 840 may include any component, device, system, or combination thereof that is configured to transmit or receive information over a network. In some embodiments, the communication unit 840 may communicate with other devices at other locations, the same location, or even other components within the same system. For example, the communication unit 840 may include a modem, a network card (wireless or wired), an optical communication device, an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, or others), and/or the like. The communication unit 840 may permit data to be exchanged with a network and/or any other devices or systems described in the present disclosure. For example, the communication unit 840 may allow the system 800 to communicate with other systems, such as computing devices and/or other networks.

One skilled in the art, after reviewing this disclosure, may recognize that modifications, additions, or omissions may be made to the system 800 without departing from the scope of the present disclosure. For example, the system 800 may include more or fewer components than those explicitly illustrated and described.

It is to be understood that when reference is made herein to choroidal imaging for use with AI, use in analysis, detecting correlations, etc., it will be appreciated that other imaging may be combined with or used in conjunction with the choroidal imaging. For example, in addition to choroidal imaging, retinal vascular imaging may also be considered or analyzed in conjunction with corresponding choroidal images. In these and other embodiments, the identification of correlations, disease-states, etc. may be based on both the choroidal imaging and the retinal vascular imaging together.

The subject technology of the present disclosure is illustrated, for example, according to various aspects described below. Various examples of aspects of the subject technology are described as numbered examples (1, 2, 3, etc.) for convenience. These are provided as examples and do not limit the subject technology. It is noted that any of the dependent examples or portions thereof may be combined in any combination, and placed into an independent example, e.g., Examples 1, 2, and 3. The other examples can be presented in a similar manner. The following is a non-limiting summary of some examples presented herein.

Example 1 includes a method including illuminating a region of a choroid of an eye of a patient with off-axis illumination from a first imaging channel. The method also includes capturing an image of the choroid using an image sensor, the off-axis illumination from the first imaging channel being off-set within the first imaging channel from the image sensor. The method additionally includes providing the captured image to a machine learning system.

Example 2 includes a method of conducting an eye exam including illuminating a region of a choroid of an eye of a patient with off-axis illumination from a first imaging channel and capturing a first image of a choroid before an intervention using an image sensor in the first imaging channel, the off-axis illumination from the first imaging channel being off-set within the first imaging channel from the image sensor. The method also includes identifying one or more first indices based on computer processing of the first image of the choroid. The method also includes capturing a second image of the choroid after the intervention using the image sensor in the first imaging channel and identifying one or more second indices based on computer processing of the second image of the choroid. The method also includes comparing the one or more first indices to the one or more second indices. The method additionally includes identifying effects of the intervention based on the comparing the one or more first indices to the one or more second indices.

Example 3 includes a method of performing choroidal imaging using a handheld imaging device including illuminating a region of a choroid of an eye of a patient with off-axis illumination from a first imaging channel and capturing a first image of a choroid using an image sensor in the first imaging channel, the off-axis illumination from the first imaging channel being off-set within the first imaging channel from the image sensor. The method also includes identifying one or more first indices based on computer processing of the first image of the choroid. The method also includes capturing a second image of the choroid using the image sensor in the first imaging channel and identifying one or more second indices based on computer processing of the second image of the choroid.

In some examples, illuminating the region of the choroid of the patient may include illuminating the region of the choroid with a second off-axis illumination from a second imaging channel that is off-axis from both the first imaging channel and the off-axis illumination.

In some examples, the first imaging channel and the off-axis illumination may capture a first image of the choroid of the eye and the second imaging channel and the second off axis-illumination capture a second image of the choroid of the eye, the first image and the second image having an overlapping region. In such examples, the first image and the second image may be combined into a single image, the single image having a wider field of view than either of the first image or the second image.

Some examples include one or more additional operations, which may include filtering out a first wavelength of light present in the captured image, the first wavelength of light being associated with a first color. In such examples, a second wavelength of light present in the captured image may be emphasized, the second wavelength of light being associated with a second color. In such examples, the second wavelength of light may be associated with a red color.

In some examples, the off-axis illumination may be a wide-spectrum light source. In such examples, the wide-spectrum light source may be a bright white light-emitting diode (LED).

In some examples, capturing the image of the choroid may include sharpening the captured image, wherein sharpening the captured image includes applying an unsharp mask to the captured image. In such examples, a sharpening radius for detecting edges may be determined, the sharpening radius corresponding to a target layer of choroidal vessels of the choroid of the eye with a target size, the sharpening radius used in sharpening the captured image.

Some examples include one or more additional operations, which may include capturing a series of images of the choroid. The series of images of the choroid may have a common focus depth and a common wavelength of illumination. The series of images of the choroid may have more than one focus depth. The series of images of the choroid may be combined to produce a video of the choroid. In such examples, the flow through choroidal vessels may be analyzed to identify a heartbeat in the video of the choroid.

Some examples include one or more additional operations, which may include identifying one or more indices based on an output of the machine learning system. The one or more indices may include at least one of average choroidal vascular caliber, average choroidal vascular tortuosity, ratio of choroidal vascular caliber to retinal vascular caliber, ratio of choroidal vascular tortuosity to retinal vascular tortuosity, categorization based on choroidal branching patterns, or choroidal vascular density. The one or more indices may be identified based on a region of the image of the choroid that is smaller than the entire image of the choroid. The machine learning system may be trained to identify at least one of specific diseases or endpoint measures based on the one or more identified indices.

It is understood that the processor may include any number of processors distributed across any number of networks or physical locations that are configured to perform individually or collectively any number of operations described herein. In some embodiments, the processor may interpret and/or execute program instructions and/or processing data stored in the memory. By interpreting and/or executing program instructions and/or process data stored in the memory, the device may perform operations, such as the operations performed by the panoramic gonioscopic apparatuses described in the present disclosure.

The memory may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor. By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. In these and other embodiments, the term “non-transitory” as used herein should be construed to exclude only those types of transitory media that were found to fall outside the scope of patentable subject matter in the Federal Circuit decision of In re Nutjten, 500 F.3d 1346 (Fed. Cir. 4007). In some embodiments, computer-executable instructions may include, for example, instructions and data configured to cause the processor to perform a certain operation or group of operations as described in the present disclosure.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method. For example, the dashed lines of the illumination paths and imaging paths are not meant to reflect an actual optical design, but are illustrative of the concepts of the present disclosure.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner. Additionally, the term “about” or “approximately” should be interpreted to mean a value within 10% of actual value.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

In addition to the color drawings, the drawings are also submitted in grayscale to facilitate understanding of the present disclosure.

1. A method comprising:
illuminating a region of a choroid of an eye of a patient with off-axis illumination from a first imaging channel, the first imaging channel being off-axis with respect to an axis of a focus of the eye;
capturing an image of the choroid using an image sensor in the first imaging channel, the off-axis illumination from the first imaging channel being off-set within the first imaging channel from the image sensor; and
providing the captured image to a machine learning system.

2. The method of claim 1, wherein illuminating the region of the choroid of the patient further comprises illuminating the region of the choroid with a second off-axis illumination from a second imaging channel that is off-axis from both the first imaging channel and the off-axis illumination.

3. The method of claim 2, wherein the first imaging channel and the off-axis illumination capture a first image of the choroid of the eye and the second imaging channel and the second off-axis illumination capture a second image of the choroid of the eye, the first image and the second image having an overlapping region, the method further comprising combining the first image and the second image into a single image, the single image having a wider field of view than either of the first image or the second image.

4. The method of claim 1, further comprising processing the captured image, comprising:
filtering out a first wavelength of light present in the captured image, the first wavelength of light associated with a first color; and
emphasizing a second wavelength of light present in the captured image, the second wavelength of light being associated with a second color.

5. The method of claim 1, wherein the second wavelength of light is associated with a red color.

6. The method of claim 1, wherein the off-axis illumination is a wide-spectrum light source.

7. The method of claim 6, wherein the wide-spectrum light source is a bright white light-emitting diode (LED).

8. The method of claim 1, further comprising sharpening the captured image by applying an unsharp mask to the captured image.

9. The method of claim 8, further comprising determining a sharpening radius for detecting edges, the sharpening radius corresponding to a target layer of choroidal vessels of the choroid of the eye with a target size, the sharpening radius used in sharpening the captured image.

10. The method of claim 1, wherein capturing the image of the choroid further comprises capturing a series of images of the choroid.

11. The method of claim 10, wherein the series of images of the choroid has a common focus depth and a common wavelength of illumination.

12. The method of claim 10, wherein the series of images of the choroid has more than one focus depth.

13. The method of claim 10, further comprising combining the series of images of the choroid to produce a video of the choroid.

14. The method of claim 13, further comprising identifying a heartbeat by analyzing flow through choroidal vessels in the video of the choroid.

15. The method of claim 1, further comprising identifying one or more indices based on an output of the machine learning system.

16. The method of claim 15, wherein the one or more indices include at least one of average choroidal vascular caliber, average choroidal vascular tortuosity, ratio of choroidal vascular caliber to retinal vascular caliber, ratio of choroidal vascular tortuosity to retinal vascular tortuosity, categorization based on choroidal branching patterns, or choroidal vascular density.

17. The method of claim 15, wherein the one or more indices may be identified based on a region of the image of the choroid that is smaller than the entire image of the choroid.

18. The method of claim 15, further comprising training the machine learning system to identify one or more specific diseases based on the one or more identified indices.

19. A method of conducting an eye exam comprising:
illuminating a region of a choroid of an eye of a patient with off-axis illumination from a first imaging channel;
capturing a first image of the choroid before an intervention using an image sensor in the first imaging channel, the off-axis illumination from the first imaging channel being off-set within the first imaging channel from the image sensor;
identifying one or more first indices based on computer processing of the first image of the choroid;
capturing a second image of the choroid after the intervention using the image sensor in the first imaging channel;
identifying one or more second indices based on computer processing of the second image of the choroid;
comparing the one or more first indices to the one or more second indices; and
identifying effects of the intervention based on the comparing the one or more first indices to the one or more second indices.

20. A method of performing choroidal imaging using a handheld imaging device, the method comprising:
illuminating a region of a choroid of an eye of a patient with off-axis illumination from a first imaging channel;
capturing a first image of the choroid using an image sensor in the first imaging channel, the off-axis illumination from the first imaging channel being off-set within the first imaging channel from the image sensor;
identifying one or more first indices based on computer processing of the first image of the choroid;
capturing a second image of the choroid using the image sensor in the first imaging channel; and
identifying one or more second indices based on computer processing of the second image of the choroid.
